(19) United States
(12) Patent Application Publication
(10) Pub. No.: US 2019/0148441 A1
(43) Pub. Date: May 16, 2019

(54) MICRO-ENDOSCOPE

(71) Applicant: Goldtek Technology Co., Ltd., New Taipei City (TW)

(72) Inventor: LI-JEN LIU, New Taipei (TW)

(21) Appl. No.: 16/185,023

(22) Filed: Nov. 9, 2018

(30) Foreign Application Priority Data
Nov. 10, 2017 (TW) 106138980

(51) Int. Cl. H01L 27/146; H01L 23/498; H04N 5/225; A61B 1/00; A61B 1/05; A61B 90/20

(52) U.S. Cl. CPC H01L 27/14636; H01L 23/49838; H04N 5/2253; H04N 2005/2255; A61B 1/051; A61B 90/20; A61B 1/00114

(57) ABSTRACT

A micro endoscope device comprises: a wafer level image capturing module, which includes a light receiving terminal configured to receive light input, thereby enabling generation of an image signal and conversion of the image signal into plurality of electric signals, and a signal in/output terminal having a plurality of electrical contacts and configured to output the electric signals; a first interposer that includes a first substrate and a plurality of electrical contacts, the first substrate having a plurality of holes defined thereon; a second interposer that includes a second substrate and a plurality of electrical contacts, the second substrate having a plurality of holes defined thereon; a micro-coaxial cable comprising a plurality of micro-wires; wherein the wafer level image capturing module is coupled to the first interposer, the first interposer is coupled to the second interposer, and the second interposer is coupled to the micro-coaxial cable.

### CROSS-REFERENCE TO RELATED APPLICATIONS

This application claims priority to Taiwan Patent Application No. 106138980, filed on Nov. 10, 2017, the contents of which are incorporated by reference herein.

### FIELD

The instant disclosure relates to a micro endoscope device.

### BACKGROUND

Medical endoscopes have been an important medical device. Micro endoscope devices are widely used in minimal invasive surgery because of their miniaturized size. However, because of their small size, micro-endoscope devices are difficult to manufacture in large scale. There is a need for continual improvement in micro endoscope devices to achieve further miniaturization and compatibility in large-scale mass production.

### SUMMARY

In view of the abovementioned needs in the technical field of medical device, the present disclosure provides a micro endoscope device that can simultaneously satisfy the demand for miniaturization and efficient mass production.

The instant disclosure provides a micro endoscope device that comprises: a wafer level image capturing module, a first interposer, a second interposer, and a micro-coaxial cable. The wafer level image capturing module includes a light receiving terminal configured to receive light input, so as to enable generation of an image signal by the wafer level image capturing module and conversion of the image signal into plurality of electric signals, and a signal in/output terminal having a plurality of electrical contacts and configured to output the electric signals. The first interposer includes a first substrate and a plurality of electrical contacts, and has a plurality of holes defined thereon. The second interposer includes a second substrate and a plurality of electrical contacts, and has a plurality of holes defined thereon. The micro-coaxial cable comprises a plurality of micro-wires. The wafer level image capturing module is coupled to the first interposer, the first interposer is coupled to the second interposer, and the second interposer is coupled to the micro-coaxial cable.

In some embodiments, the wafer level image capturing module is electrically connected to the first interposer, the first interposer is electrically connected to the second interposer, and the second interposer is electrically coupled to the micro-coaxial cable.

In some embodiments, the wafer level image capturing module is electrically connected to the first interposer, the first interposer is electrically connected to the second interposer, and the second interposer is electrically coupled to the micro-coaxial cable.

In some embodiments, the micro-endoscope further comprises a hollow tube that defines an accommodating compartment that receives the wafer level image capturing module, the first interposer board, the second interposer, and a portion of the micro cable.

In some embodiments, each of the electrical contacts of the signal output terminal of the wafer level image capturing module respectively comprises a solder connector.

In some embodiments, each of the electrical contacts of the first interposer respectively comprises a solder connector.

In some embodiments, each of the electrical contacts of the second interposer respectively comprises a solder connector.

In some embodiments, the first interposer is one of a glass substrate, a ceramic substrate, or a silicon substrate.

In some embodiments, the hollow tube is a hollow steel tube or a hollow plastic tube.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a side view of a micro-endoscope device in accordance with one embodiment the present disclosure.

FIG. 2A is a side view of a wafer level image capturing module of the micro endoscope device according to one embodiment of the present disclosure.

FIG. 2B is a schematic diagram of a signal output terminal of a wafer level image capturing module of the micro endoscope device according to an embodiment of the present disclosure.

FIG. 3A is a side view of the first interposer of a micro endoscope device according to an embodiment of the present disclosure.

FIG. 3B is a schematic illustration of a first input terminal of a first interposer of a micro-endoscope device in accordance with an embodiment of the present disclosure.

FIG. 3C is a schematic view showing the first output terminal of the first interposer of a micro endoscope device according to one embodiment of the present disclosure.

FIG. 4A is a side view of the second interposer of a micro endoscope device according to one embodiment of the present disclosure.

FIG. 4B is a schematic view showing the second input terminal of the second interposer of the micro endoscope device according to one embodiment of the present disclosure.

FIG. 4C is a schematic view showing the second output terminal of the second interposer of the micro endoscope device according to one embodiment of the present disclosure.

FIG. 5 is a schematic view of a micro-coaxial cable of a micro endoscope device according to one embodiment of the present disclosure.

It is to be noted, however, that the appended drawings illustrate only exemplary embodiments of this disclosure and are therefore not to be considered limiting of its scope, for the disclosure may admit to other equally effective embodiments.

### DETAILED DESCRIPTION

The present disclosure will now be described more fully hereinafter with reference to the accompanying drawings, in which exemplary embodiments of the disclosure are shown. This disclosure may, however, be embodied in many different forms and should not be construed as limited to the exemplary embodiments set forth herein. Rather, these exemplary embodiments are provided so that this disclosure will be thorough and complete, and will fully convey the scope of the disclosure to those skilled in the art. Like reference numerals refer to like elements throughout.

The terminology used herein is for the purpose of describing particular exemplary embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

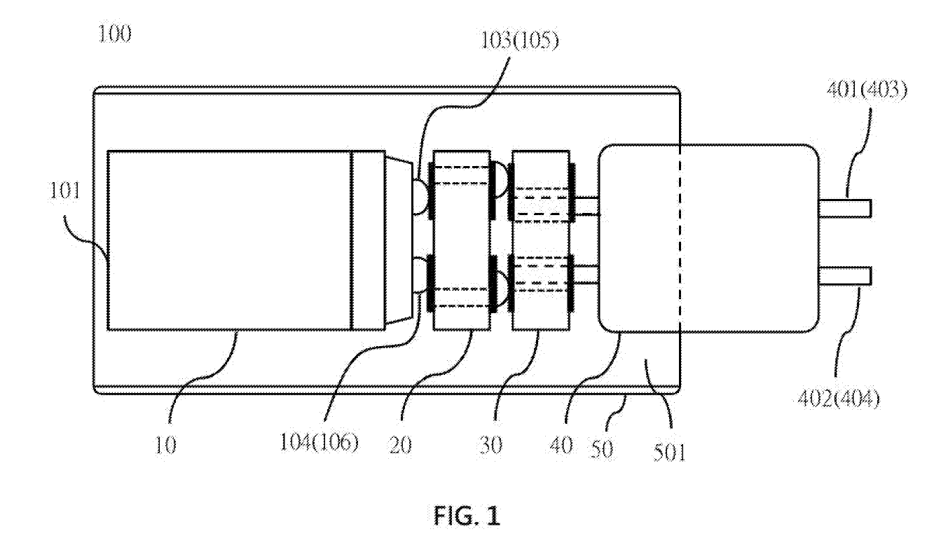

The description will be made as to the exemplary embodiments in conjunction with the accompanying drawings in FIGS. 1 to 5. Reference will be made to the drawing figures to describe the present disclosure in detail, wherein depicted elements are not necessarily shown to scale and wherein like or similar elements are designated by same or similar reference numeral through the several views and same or similar terminology.

FIG. 1 is a side view of a micro-endoscope device in accordance with one embodiment the present disclosure. The micro endoscope device 100 includes a wafer-level image capturing module 10, a first interposer 20, a second interposer 30, a micro-coaxial cable 40, and a hollow tube 50. The wafer level image capturing module 10 is structurally and electrically connected to the first interposer 20. The first interposer 20 is structurally and electrically connected to the second interposer 30. The second interposer 30 is further connected to the micro-coaxial cable 40. and electrically coupled thereto. The wafer level image capturing module 10, the first interposer 20, the second interposer 30, and a portion of the micro-coaxial cable 40 are arranged in an accommodating space 501 defined by the hollow tube 50. The accommodating space 501 is further filled with silicone (not shown) to provide moisture prevention and shock absorption. The wafer level image capturing module 10 includes a light receiving terminal 101, a wafer-level camera module (not shown), and a complementary metal oxide semiconductor (CMOS) device (not shown), solder connectors 103-106 (where solder connector 105 and 106 are blocked from current view). The micro-coaxial cable 40 includes four micro-wires 401, 402, 403, and 404 (where the micro-wires 403 and 404 are blocked from instant view). The hollow tube 50 may include a hollow steel tube or a hollow plastic tube. The complementary metal oxide semiconductor device (not shown) may include a charge coupled-capacitor (CCD).

During operation, a light beam (not shown) reflected from a target object (not shown) is incident toward the wafer level image capturing module 10 via the light receiving terminal 101. The incident light beam (not shown) passes through a wafer-level lens module (not shown) and is focused and imaged on a complementary metal oxide semiconductor (CMOS) device (not shown). The CMOS device captures an image signal (not shown), and converts the signal into a corresponding electrical signal (not shown). The electrical signal may then be output to the first interposer 20 through the solder connector of the signal input/output (I/O) terminal. The first interposer 20 subsequently outputs the electrical signal to the second interposer 30, and the second interposer 30 in turn outputs the electrical signal to the micro-coaxial cable 40. The electrical signal may finally be output by the micro-wires in the micro-coaxial cable 40. The above complementary metal oxide semiconductor may be a charge coupled device (CCD).

The structural and electrical arrangement between the wafer-level image capturing module 10, the first interposer 20, the second interposer 30, and the micro-coaxial cable 40 will be described in more detail as follows.

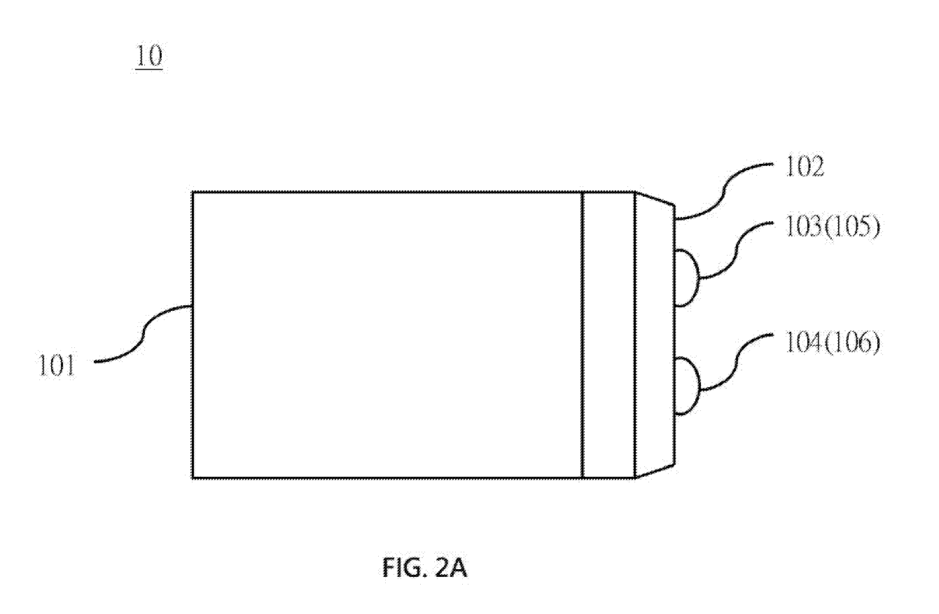

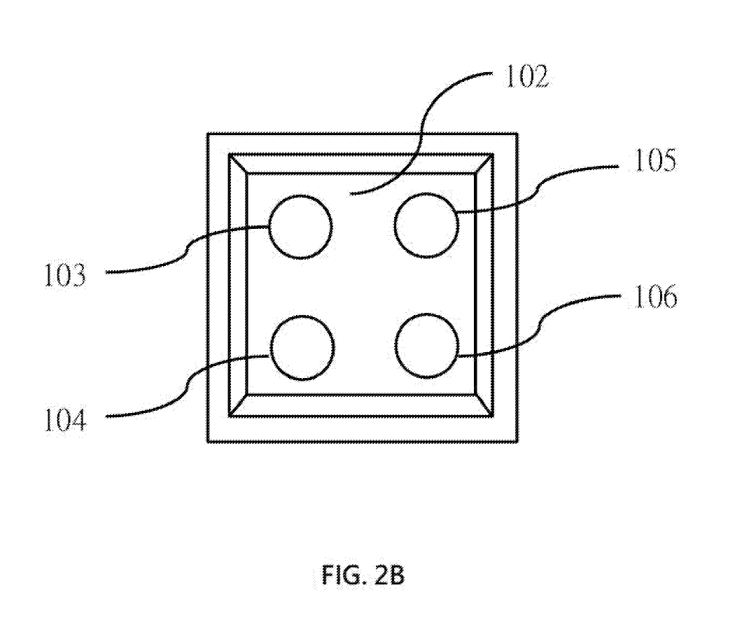

Please refer to FIG. 2A and FIG. 2B concurrently. FIG. 2A shows a side view of a wafer level image capturing module of the micro endoscope device according to one embodiment of the present disclosure, and FIG. 2B illustrates a schematic diagram of a signal output terminal of a wafer level image capturing module of the micro endoscope device according to an embodiment of the present disclosure. The wafer level image capturing module 10 includes a light receiving terminal 101, a signal input/output (I/O) terminal 102, solder connectors (e.g., solder balls) 103-106. The signal output terminal 102 includes four electrical contacts (not visible in current view due to coverage by the solder balls). The solder connectors 103-106 are respectively attached to the signal input/output terminal 102.

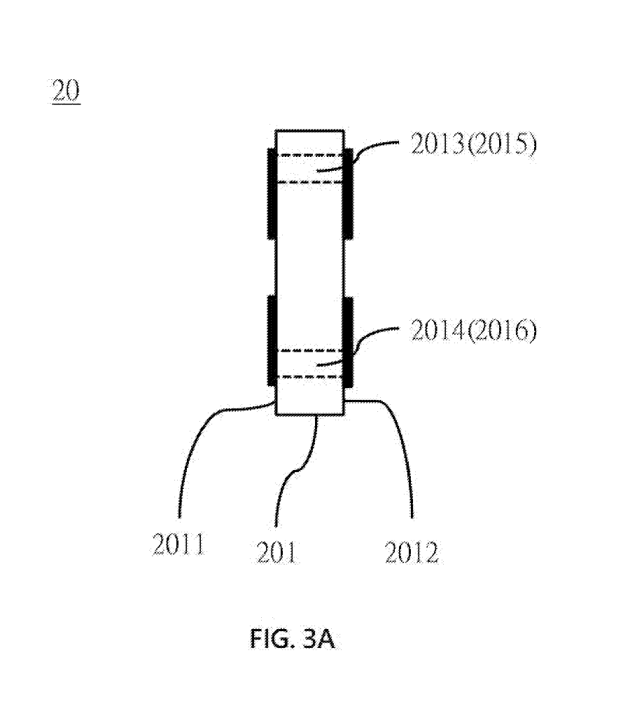

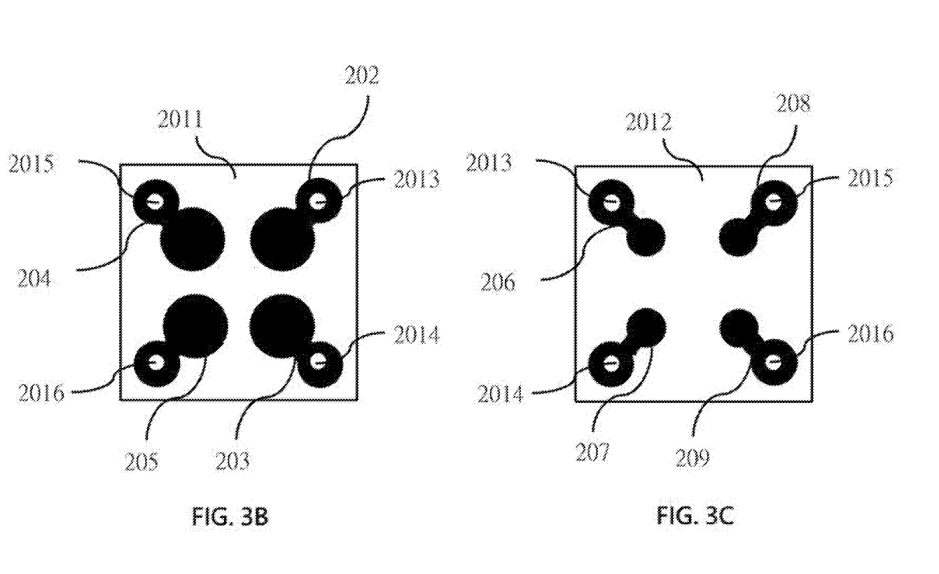

Please refer to FIG. 3A, FIG. 3B and FIG. 3C concurrently. FIG. 3A shows a side view of the first interposer of a micro endoscope device according to an embodiment of the present disclosure. FIG. 3B provides a schematic illustration of a first input terminal of a first interposer of a micro-endoscope device in accordance with an embodiment of the present disclosure. FIG. 3C is a schematic view showing the first output terminal of the first interposer of a micro endoscope device according to one embodiment of the present disclosure. The first interposer 20 includes a first substrate 201, electrical contacts 202-209. The first substrate 201 further has through holes 2013-2016 defined thereon, each of which penetrates the substrate 201 and respectively enables electrical connection between the corresponding electrical contacts 202-209 on the two sides (the first input end 2011 and the first output end 2012) of the first substrate 201. In the instant embodiment, the electrical contacts 201-205 are arranged on the first input end (side) 2011, while electrical contacts 206-209 are arranged on the first output end (side) 2012 of the first substrate 201. Suitable material for the first substrate 201 may include glass, ceramic, and silicon.

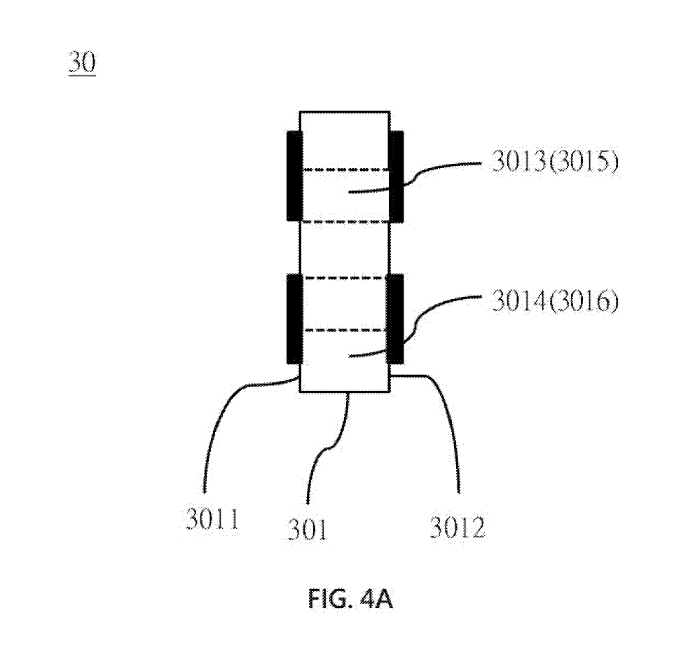

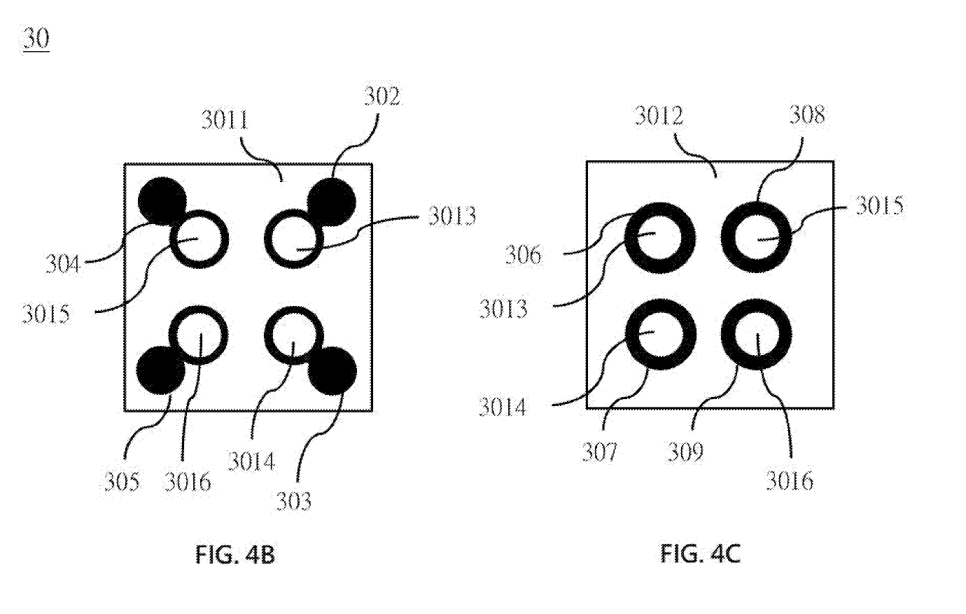

Please concurrently refer to FIG. 4A, FIG. 4B and FIG. 4C. FIG. 4A is a side view of the second interposer of a micro endoscope device according to one embodiment of the present disclosure. FIG. 4B is a schematic view showing the second input terminal of the second interposer of the micro endoscope device according to one embodiment of the present disclosure. FIG. 4C is a schematic view showing the second output terminal of the second interposer of the micro endoscope device according to one embodiment of the present disclosure. The second interposer 30 includes a second substrate 301 and electrical contacts 302-309. The second substrate 301 further has through holes 3013-3016 defined thereon. Each of the through holes 3013-3016 penetrates the second substrate 301 and enables electrical connection between the corresponding electrical contacts 302-309 respectively arranged on the two opposing sides (the second input end 3011 and the second output end 3012) of the second substrate 301. The second substrate 301 may be glass, ceramic, or silicon substrate.

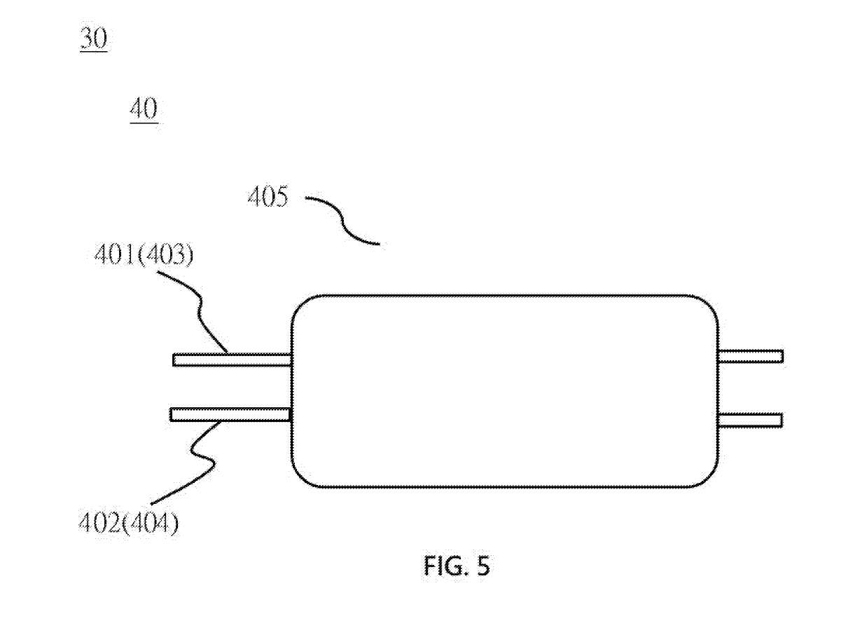

Please refer to FIG. 5, which shows a schematic view of a micro-coaxial cable of a micro endoscope device according to one embodiment of the present disclosure. The micro-coaxial cable 40 includes micro-wires 401-404 (where micro-wires 403 and 404 are obstructed from instant view), and a cladding layer 405.

During assembling of the endoscope device, the first input terminal 2011 of the first interposer 20 (as shown in FIG. 3B) is first brought in contact with the signal output terminal 102 of the wafer level image capturing module 10 (as shown in FIG. 2B), so that the solder connectors 103-106 (as shown in FIG. 2B) respectively establish contact with the electrical contacts 202-205 (as shown in FIG. 3B). A reflow process is subsequently performed to melt the solder material of the connectors, thereby causing the filling of solder material into the through holes 2013-2016 (as shown in FIGS. 3B and 3C). In some embodiments, excessive solder material may over-flow/ooze from the through holes and reach the respective electrical contacts 206-209 (as shown in FIG. 3C). Upon solidification of the solder material, the wafer level image capturing module 10 will establish structural connection with the first interposer 20, while the solder connectors 103-106 will establish electrical connection with the electrical contacts 206-209, respectively.

The micro-wires 401, 402, 403, and 404 of the micro-coaxial cable 40 (refer to FIG. 5) are respectively inserted into the through holes 3013-3016 in the second substrate 30 from the second output end 3012. Solder materials are subsequently placed on the electrical contacts 206-209 of the first interposer 20 on the first output end/side 2012. Likewise, the electrical contacts 302-305 on the second input end/side 3011 of the second interposer 30 are brought in contact with the electrical contacts 206-209 on the first output end/side 2012 of the first interposer 20 (as shown in FIG. 3C), respectively. A heating process is then carried out, causing reflow of the solder material and thereby filling the through holes 3013-3016 (referring to FIGS. 4B and 4C). In some embodiments, excessive solder material may exude from the through holes and reach the respective electrical contacts 306-309 (as shown in FIG. 4C). Upon solidification of the solder material, the second interposer 30 will respectively establish structural connection with the first interposer 20 and the micro-coaxial cable 40. The electrical contacts 206-209 will establish electrical connection with micro-wires 401-404 through the electrical contacts 302-305 and 306-309. The electrical connection between the wafer level image capturing module 10 and the micro-coaxial cable 40 is thereby provided through the respective connection between the solder connectors 103-106 of the wafer-level image capturing module 10 and the micro-wires 401, 402, 403 and 404 of the micro-coaxial cable 40.

In the above exemplary embodiment, solder connectors are not pre-attached to the electrical contacts of the first and the second interposers 20, 30. However, in some embodiments, the respective electrical contacts of the interposers may be provided with solder material in advance. It should be noted that, such variation should also be encompassed within the scope of this disclosure.

Likewise, in the above exemplary embodiment, the signal input/output terminals of the wafer level image capturing module 10 are provided with pre-attached solder materials. However, it should be noted that, in some embodiment, the signal input/output terminals of the wafer level image capturing module may be provided without pre-attachment of solder material. Such variation should also be encompassed within the scope of this disclosure.

Accordingly, embodiments of the instant disclosure provide a micro endoscope device that comprises a wafer level image capturing module, a first interposer, a second interposer, and a micro-coaxial cable. The wafer level image capturing module includes a light receiving terminal configured to receive light input, so as to enable generation of an image signal by the wafer level image capturing module and conversion of the image signal into plurality of electric signals, and a signal in/output terminal having a plurality of electrical contacts and configured to output the electric signals. The first interposer includes a first substrate and a plurality of electrical contacts, and has a plurality of holes defined thereon. The second interposer includes a second substrate and a plurality of electrical contacts, and has a plurality of holes defined thereon. The micro-coaxial cable comprises a plurality of micro-wires. The wafer level image capturing module is coupled to the first interposer, the first interposer is coupled to the second interposer, and the second interposer is coupled to the micro-coaxial cable.

The embodiments shown and described above are only examples. Even though numerous characteristics and advantages of the present technology have been set forth in the foregoing description, together with details of the structure and function, the disclosure is illustrative only, and changes may be made in the detail, especially in matters of shape, size, and arrangement of the parts within the principles, up to and including the full extent established by the broad general meaning of the terms used in the claims. It will therefore be appreciated that the embodiments described above may be modified within the scope of the claims.

What is claimed is:

1. A micro endoscope device, comprising:
a wafer level image capturing module, which includes
a light receiving terminal configured to receive light input, so as to enable generation of an image signal by the wafer level image capturing module and conversion of the image signal into plurality of electric signals, and
a signal in/output terminal having a plurality of electrical contacts and configured to output the electric signals;
a first interposer that includes a first substrate and a plurality of electrical contacts, the first substrate having a plurality of holes defined thereon;
a second interposer that includes a second substrate and a plurality of electrical contacts, the second substrate having a plurality of holes defined thereon;
a micro-coaxial cable comprising a plurality of micro-wires;
wherein the wafer level image capturing module is coupled to the first interposer, the first interposer is coupled to the second interposer, and the second interposer is coupled to the micro-coaxial cable.

2. The device of claim 1, wherein the wafer level image capturing module is electrically connected to the first interposer, the first interposer is electrically connected to the second interposer, and the second interposer is electrically coupled to the micro-coaxial cable.

3. The device of claim 1, further comprising a hollow tube that defines an accommodating compartment that receives the wafer level image capturing module, the first interposer board, the second interposer, and a portion of the micro cable.

4. The device according to claim 1, wherein each of the electrical contacts of the signal output terminal of the wafer level image capturing module respectively comprises a solder connector.

5. The device of claim 1, wherein each of the electrical contacts of the first interposer respectively comprises a solder connector.

6. The device of claim 1, wherein each of the electrical contacts of the second interposer respectively comprises a solder connector.

7. The device of claim 1, wherein the first interposer is one of a glass substrate, a ceramic substrate, or a silicon substrate.

8. The device according to claim 3, wherein the hollow tube is a hollow steel tube or a hollow plastic tube.
